(19) United States
(12) Patent Application Publication
(10) Pub. No.: US 2023/0145460 A1
(43) Pub. Date: May 11, 2023

(54) STEERABLE MEDICAL DEVICE, HANDLE FOR A MEDICAL DEVICE, AND METHOD FOR OPERATING A MEDICAL DEVICE

(71) Applicant: Boston Scientific Medical Device Limited, Galway (IE)

(72) Inventors: James Klein, Mississauga (CA); Jan-Hung Chen, Mississauga (CA)

(21) Appl. No.: 18/151,318

(22) Filed: Jan. 6, 2023

Related U.S. Application Data
(63) Continuation of application No. PCT/IB2021/056055, filed on Jul. 6, 2021.
(60) Provisional application No. 63/049,202, filed on Jul. 8, 2020.

(51) Int. Cl. A61M 25/01

(52) U.S. Cl. CPC A61M 25/0136; A61M 25/0113

(57) ABSTRACT

A handle for a medical device includes a rotatable knob assembly and a slide assembly. The knob assembly is couplable to the slide assembly to drive movement of the slide assembly by rotation of the knob assembly. A slip clutch assembly is between the knob and slide assemblies. The slip clutch assembly includes a first disc and a second disc. The first disc is secured to the knob assembly, and is rotatable with the knob assembly. The second disc is configured to transmit rotation to the slide assembly. The first disc and/or the second disc is translatable between a first position and a second position. In the first position, the first and second discs are moved into engagement to couple the rotatable knob to the slide assembly. In the second position, the first and second discs are moved out of engagement to decouple the rotatable knob from the slide assembly.

### CROSS REFERENCE TO RELATED APPLICATION

This application is a continuation application of International Application No. PCT/IB2021/056055, filed Jul. 6, 2021, titled “STEERABLE MEDICAL DEVICE, HANDLE FOR A MEDICAL DEVICE, AND METHOD FOR OPERATING A MEDICAL DEVICE,” which claims priority to U.S. Provisional Application No. 63/049,202, filed Jul. 8, 2020, titled “STEERABLE MEDICAL DEVICE, HANDLE FOR A MEDICAL DEVICE, AND METHOD FOR OPERATING A MEDICAL DEVICE,” the entire disclosures of which are incorporated herein by reference.

### FIELD

This document relates to medical devices. More specifically, this document relates to steerable medical devices such as steerable sheaths, catheters, and introducers.

### SUMMARY

The following summary is intended to introduce the reader to various aspects of the detailed description, but not to define or delimit any invention.

Steerable medical devices are disclosed. According to some aspects, a steerable medical device includes a handle having a rotatable knob assembly and housing a slide assembly. The knob assembly is couplable to the slide assembly to drive movement of the slide assembly by rotation of the knob assembly. An elongate tool extends from the handle. At least one control wire is coupled between the slide assembly and the tool. Movement of the slide assembly causes tensioning of the control wire, and tensioning of the control wire causes deflection of the tool. A slip clutch assembly is between the knob assembly and the slide assembly. When torque applied to the knob assembly is below a threshold value, the slip clutch assembly couples the knob assembly to the slide assembly to drive movement of the slide assembly by rotation of the knob assembly. When torque applied to the knob assembly is above the threshold value, the slip clutch assembly decouples the knob assembly from the slide assembly to prevent movement of the slide assembly by rotation of the knob assembly.

In some examples, the slip clutch assembly includes a first disc rotatable with the knob assembly and having a first engagement face, and a second disc configured to transmit rotation to the slide assembly and having a second engagement face. At least one of the first disc and the second disc can translate between a first position and a second position. In the first position, the first engagement face and the second engagement face are engaged to couple the rotatable knob to the slide assembly. In the second position the first engagement face and the second engagement face are disengaged to decouple the rotatable knob from the slide assembly.

In some examples, the first disc is fixed with respect to the knob assembly, and the second disc is coupled to the slide assembly and is translatable between the first position and the second position.

In some examples, the slip clutch assembly further includes a driveshaft fixed to the slide assembly and extending towards the rotatable knob. The driveshaft can include a plurality of splines. The second disc can be annular and can include an inner surface having a plurality of longitudinal grooves. The second disc can be received on the drive shaft with the splines engaged with the grooves.

In some examples, the slip clutch assembly further includes a spring biasing the second disc towards the first position. The slip clutch assembly can further include a flange from which the driveshaft extends. The spring can be received on the driveshaft and can be compressed between the flange and the second disc.

In some examples, the first engagement face has a first set of teeth, and the second engagement face has a second set of teeth, and the first set of teeth and the second set of teeth each have inclined side surfaces to facilitate movement from the first position to the second position by rotation of the first disc.

In some examples, the second disc is longitudinally fixed with respect to the first disc. When torque applied to the knob assembly is below a threshold value, the first disc can transmit rotation to the second disc by magnetic forces or frictional forces between the first engagement face and the second engagement face.

In some examples, the tool is a sheath, a catheter, or an introducer.

Handles for medical devices are also disclosed. According to some aspects, a handle for a medical device includes a rotatable knob assembly and houses a slide assembly. The knob assembly is couplable to the slide assembly to drive movement of the slide assembly by rotation of the knob assembly. A slip clutch assembly is between the knob assembly and the slide assembly. The slip clutch assembly includes a first disc and a second disc. The first disc has a first engagement face and is rotatable with the knob assembly. The second disc has a second engagement face and is configured to transmit rotation to the slide assembly. The first engagement face and the second engagement face are engageable to couple the rotatable knob to the slide assembly to drive movement of the slide assembly by rotation of the knob assembly, and are disengageable to decouple the rotatable knob and the slide assembly to prevent movement of the slide assembly by rotation of the knob assembly.

In some examples, at least one of the first disc and the second disc is translatable with respect to the slide assembly between a first position and a second position. In the first position, the first engagement face and the second engagement face can be engaged to couple the rotatable knob to the slide assembly. In the second position, the first engagement face and the second engagement face can be disengaged to decouple the rotatable knob from the slide assembly.

In some examples, the first disc is fixed to the knob assembly, and the second disc is coupled to the slide assembly and is translatable between the first position and the second position.

In some examples, the slip clutch assembly further includes a driveshaft fixed to the slide assembly and extending towards the knob assembly. The driveshaft can include a plurality of splines. The second disc can be annular and can include an inner surface having a plurality of longitudinal grooves. The second disc can be received on the drive shaft with the splines engaged with the grooves.

In some examples, the slip clutch assembly further includes a spring biasing the second disc towards the first position. The slip clutch assembly can further include a flange from which the driveshaft extends, and the spring can be received on the driveshaft and be compressed between the flange and the second disc.

In some examples, the first engagement face has a first set of teeth and the second engagement face has a second set of teeth. The first set of teeth and the second set of teeth can each have inclined side surfaces to facilitate translation from the first position to the second position by rotation of the first disc.

In some examples, the second disc is longitudinally fixed with respect to the first disc. The first engagement face and second engagement face can be engageable by magnetic forces or frictional forces.

Methods for operating medical devices are also disclosed. According to some aspects, a method for operating a medical device includes: a. with a knob assembly of the medical device coupled to a slide assembly of the medical device via a slip clutch assembly, applying torque to the knob assembly to rotate the knob assembly; and b. transmitting rotation of the knob assembly to the slide assembly via the slip clutch assembly.

In some examples, the slip clutch assembly includes a first disc having a first engagement face and a second disc having a second engagement face engagable with the first engagement face. The first disc can be rotatable with the knob assembly, and the second disc can be configured to transmit rotation to the slide assembly. Step b. can include transmitting rotation of the knob assembly to the first disc, transmitting rotation of the first disc to the second disc, and transmitting rotation of the second disc to the slide assembly.

In some examples, the second engagement face is maintained in engagement with the first engagement face by a spring. In some examples, the second engagement face is maintained in engagement with the first engagement face by frictional forces or magnetic forces.

In some examples, the method further includes: c. increasing the torque applied to the knob assembly to exceed a threshold value; and d. as a result of step c., decoupling the knob assembly from the slide assembly via the slip clutch assembly.

In some examples, the slip clutch assembly includes a first disc having a first engagement face and a second disc having a second engagement face engagable with the first engagement face. The first disc can be rotatable with the knob assembly, and the second disc can be configured to transmit rotation to the slide assembly. Step d. can include moving the second disc away from the first disc to disengage the first engagement face and the second engagement face. Moving the second disc away from the first disc can include translating the first disc along a driveshaft of the slip clutch assembly to compress a spring.

In some examples, the method further includes: e. decreasing the torque applied to the knob assembly to fall below the threshold value; and f. as a result of step e., recoupling the knob assembly to the slide assembly via the slip clutch assembly.

In some examples, the slip clutch assembly includes a first disc having a first engagement face and a second disc having a second engagement face engagable with the first engagement face. The first disc can be rotatable with the knob assembly, and the second disc can be configured to transmit rotation to the slide assembly. Step f. can include moving the second disc towards the first disc to engage the first engagement face and the second engagement face.

### BRIEF DESCRIPTION OF THE DRAWINGS

The accompanying drawings are for illustrating examples of articles, methods, and apparatuses of the present disclosure and are not intended to be limiting. In the drawings:

FIG. 1 is a perspective view of an example medical device;

FIG. 2 is a cutaway plan view of the handle of the medical device of FIG. 1;

FIG. 3 is an enlarged plan view of a slip clutch assembly of the handle of FIG. 2 showing coupling of a knob assembly to a slide assembly by the slip clutch assembly;

FIG. 4 is an enlarged plan view similar to that of FIG. 3, showing decoupling of the knob assembly from the slide assembly by the slip clutch assembly;

FIG. 5 is an enlarged plan view similar to that of FIG. 4, showing recoupling of the knob assembly to the slide assembly by the slip clutch assembly;

FIG. 6 is a partial exploded view of the knob assembly, slip clutch assembly, and slide assembly of the handle of FIG. 2; and

FIG. 7 is a bottom view of the second ring of FIG. 6.

### DETAILED DESCRIPTION

Various apparatuses or processes or compositions will be described below to provide an example of an embodiment of the claimed subject matter. No example described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

Generally disclosed herein are steerable medical devices that include a handle and a tool such as a sheath, a catheter, or an introducer. The handle can enable the user to manipulate or steer the tool in a desired direction. More specifically, the handle can include a knob assembly that is rotatably coupled to a housing of the handle. In operation, the rotation of the knob assembly in a first rotational direction can allow the user to steer or deflect the tool in a first direction, whereas the rotation of the knob assembly in a second rotational direction can allow the user to steer or deflect the tool in a second direction. The rotation of the knob can be converted into a deflection of the tool via a slide assembly, which can be within the housing, and one or more control wires, which are connected between the slide assembly and the tool. The slide assembly can include a threaded shaft (also referred to as a bolt), and a slider (also referred to as a carriage) that is slidable on the threaded shaft. Rotation of the knob can cause rotation of the threaded shaft, which can cause linear translation of the slider along the threaded shaft. This translation of the slider causes a tensioning of the control wire(s), which results in a deflection of the tool. For simplicity, details of the slider and the control wires are not disclosed herein. However, related sliders and control wires are disclosed in, for example, U.S. Pat. No. 10,661,057 (Davies et al.), which is incorporated herein by reference in its entirety. Furthermore, steerable medical devices including sliders and control wires are sold Baylis Medical Company, Inc. (Montreal, Canada) under the brand name SureFlex® Steerable Guiding Sheath.

The steerable medical devices disclosed herein are configured to avoid or prevent or reduce the risk of failure due to excessive tension being applied to the control wire(s). Such excessive tension can occur, for example, if a stiff secondary tool is within the tool, preventing deflection of the tool, but a user attempts to force rotation of the knob assembly. Such excessive tension can further occur if the tool is caught on patient anatomy and thus cannot deflect, but a user attempts to force rotation of the knob assembly. Such excess tension can further occur if force is applied to the tool without rotating the knob, for example if the tool is pressed by patient anatomy.

In the devices disclosed herein, to avoid or prevent or reduce the risk of failure due to excessive tension being applied to the control wire(s), a slip clutch assembly is provided between the knob assembly and the slide assembly. In use, when a relatively low amount of tension is on the control wires and torque is applied to the knob assembly (i.e. torque of below a threshold value), the knob assembly rotates, and rotation of the knob assembly causes rotation of the slide assembly via the slip clutch assembly, which couples the knob assembly and the slide assembly; however, when the amount of tension on the control wires is relatively high and torque is applied to the knob assembly (i.e. torque of above the threshold value), the slip clutch assembly decouples the knob assembly from the slide assembly, to prevent rotation of the slide assembly with the knob assembly, and thereby prevent additional tension from being applied to the control wires. This can in turn avoid or prevent or reduce the risk of breaking of the control wires or disconnection of the joint between the control wires and the slide mechanism and/or tool. Furthermore, this can avoid or prevent or reduce the risk of tissue damage if the tool is caught on patient anatomy.

As used herein, the term “slip clutch assembly” refers to an assembly that couples a first element (e.g. a knob assembly) to a second element (e.g. a slide assembly) to transmit rotation from the first element to a second element when the torque applied to the first element is below a threshold value, and decouples the first element and the second element to prevent transmission of rotation from the first element to the second element when the torque applied to the second element is above a threshold value.

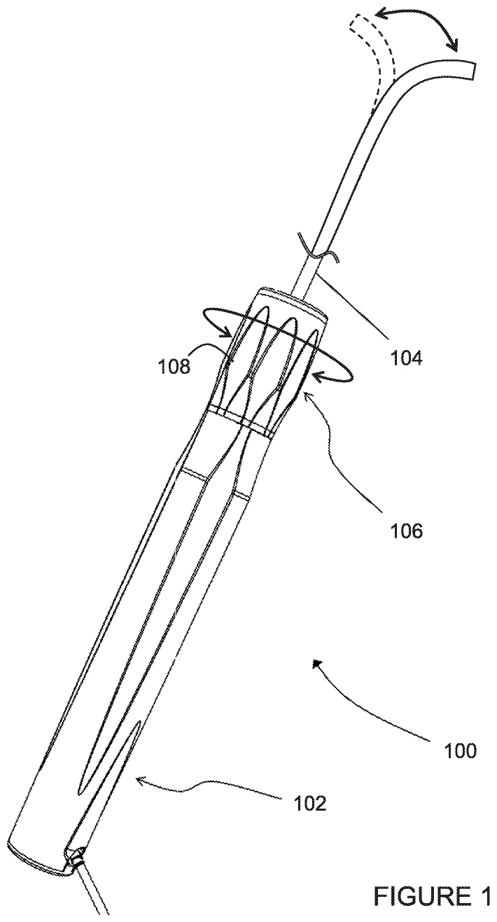

Referring now to FIG. 1, an example steerable medical device 100 is shown. The steerable medical device 100 generally includes a handle 102 and an elongate tool 104 extending from the handle. The tool 104 can be, for example (but not limited to), a sheath, a catheter, or an introducer. The handle 102 includes a rotatable knob assembly 106, which can be rotated to steer the tool 104. Rotation of the knob assembly 106 in a first direction (e.g. clockwise) can cause the tool 104 to deflect in a first direction (i.e. to the configuration shown in dotted line in FIG. 1), and rotation of the knob assembly 106 in a second direction (e.g. counter-clockwise) can cause the tool 104 to deflect in a second direction (i.e. back to the configuration shown in solid line in FIG. 1).

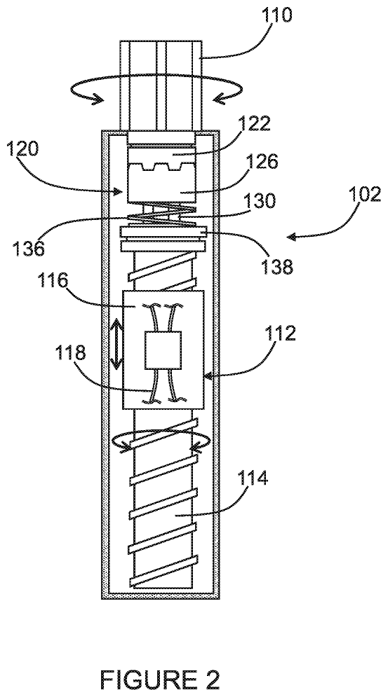

Referring still to FIG. 1 and also to FIG. 2, the knob assembly 106 includes an outer knob 108 (shown in FIG. 1), which is grasped and manipulated by the user, and an inner knob 110 (shown in FIG. 2) which is rotated by rotation of the outer knob 108.

Referring still to FIG. 2, a slide assembly 112 is housed within the handle 102. As will be described in further detail below, the knob assembly 106 (only the inner knob 110 of which is shown in FIG. 2) is couplable to the slide assembly 112 to drive movement of the slide assembly 112 by rotation of the knob assembly 106. More specifically, the slide assembly 112 includes a threaded shaft 114 and a slider 116 that is received on the threaded shaft 114 and is translatable along the threaded shaft 114. When the knob assembly 106 is coupled to the slide assembly 112, rotation of the knob assembly 106 causes rotation of the threaded shaft 114. Rotation of the threaded shaft 114 causes translation of the slider 116 along the threaded shaft 114. A control wire 118 is in turn coupled between the slider 116 of the slide assembly 112 and the tool 104 (shown in FIG. 1). Movement of the slide assembly 112—i.e. translation of the slider 116 caused by rotation of the threaded shaft 114—causes tensioning of the control wire 118, and tensioning of the control wire 118 causes deflection of the tool 104. As mentioned above, details of the slider 116 and control wire 118, and the connection between the slider 116, control wire 118 and tool 104 are disclosed in U.S. Pat. No. 10,661,057 (Davies et al.), and are not repeated herein.

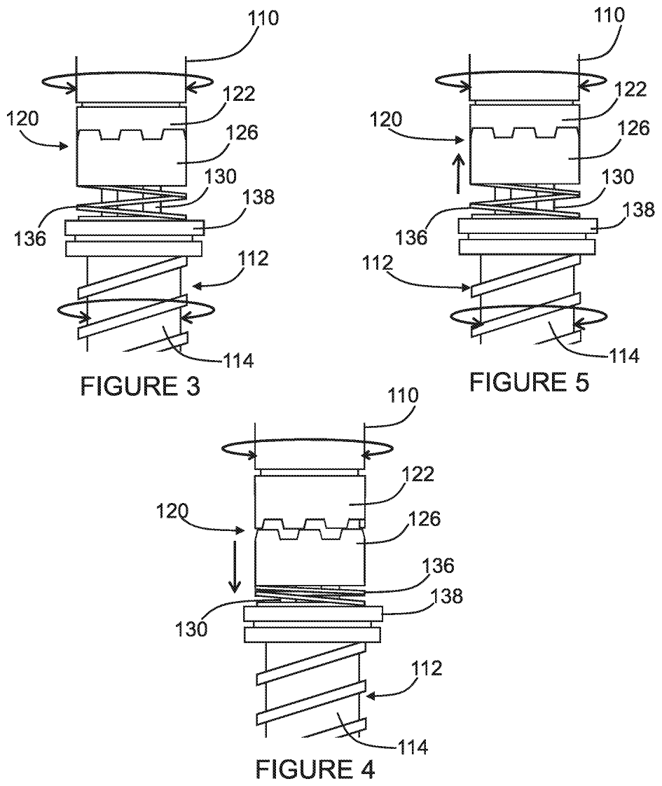

Referring to FIGS. 3 to 5, a slip clutch assembly 120 is between the knob assembly 106 (only the inner knob 110 of which is shown in FIGS. 3 to 5) and the slide assembly 112. As shown in FIG. 3, when torque applied to the knob assembly 106 is below a threshold value (e.g. when the control wire 118, not shown in FIGS. 3 to 5, is under a relatively low amount of tension, as would occur during routine use), the slip clutch assembly 120 couples the inner knob 110 of the knob assembly 106 to the slide assembly 112 to drive movement of the slide assembly 112 by rotation of the knob assembly 106. As shown in FIG. 4, when torque applied to the knob assembly 106 is above a threshold value (e.g. when the control wire 118 is under relatively high tension and rotation of the knob assembly 106 is continued), the slip clutch assembly 120 decouples the inner knob 110 of the knob assembly 106 from the slide assembly 112 to prevent movement of the slide assembly 112 by rotation of the knob assembly 106. This in turn prevents further tensioning of the control wire 118, which in turn avoids or prevents or minimizes the risk of failure. As shown in FIG. 5, when the torque applied to knob assembly 106 is lowered to below threshold value, the slip clutch assembly 120 recouples the inner knob 110 of the knob assembly 106 to the slide assemblyl12, to again drive movement of the slide assembly 112 by rotation of the knob assembly 106.

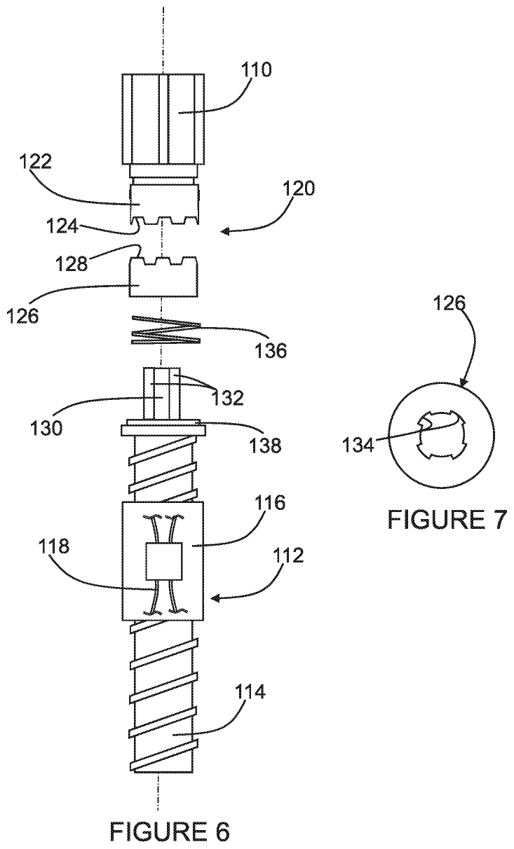

Referring to FIG. 6, in the example shown, the slip clutch assembly 120 includes a first disc 122. The first disc 122 is fixed to the knob assembly 106 (only the inner knob 110 of which is shown in FIG. 6) and is rotatable with the knob assembly 106. More specifically, in the example shown, the first disc 122 is integral with the inner knob 110. The first disc 122 has a first engagement face 124—i.e. a face that has a feature (e.g. a mechanical features such as teeth, slots, and/or bumps, and/or a magnetic feature, and/or a frictional feature) that allows for the transmission of rotation to another element. In the example shown, the first engagement face 124 includes a first set of teeth. The slip clutch assembly 120 further includes a second disc 126. The second disc 126 is mounted to the slide assembly 112 (as will be described in further detail below) to transmit rotation to the slide assembly 112. The second disc 126 has a second engagement face 128—i.e. a face that has a feature (e.g. a mechanical feature such as teeth, slots, and/or bumps, and/or a magnetic feature, and/or a frictional feature) that allows for the transmission of rotation from another element. In the example shown, the second engagement face 128 has a second set of teeth. The first engagement face 124 and second engagement face 128 face towards each other and the first set of teeth is engagable with the second set of teeth to lock the first disc 122 to the second disc 126 to drive rotation of the second disc 126 by rotation of the first disc 122 (as shown in FIG. 3). Furthermore, the first set of teeth and the second set of teeth each have inclined side surfaces to facilitate disengagement of the first set of teeth and second set of teeth by rotation of the first disc 122, as will be described below.

Referring back to FIGS. 3 to 5, the second disc 126 is translatable with respect to the slide assembly 112, between a first position (shown in FIGS. 3 and 5), and a second position (shown in FIG. 4). In the first position, the second disc 126 is moved towards the first disc 122 and into toothed engagement with the first disc 122, to couple the first disc 122 to the second disc 126 and thereby couple the knob assembly 106 to the slide assembly 112. In in the second position, the second disc 126 is moved away from the first disc 122 and out of toothed engagement with the first disc 122, to disengage the first disc 122 and the second disc 126 and thereby decouple the knob assembly from the slide assembly 112.

Referring back to FIG. 6, in the example shown, in order to allow for translation of the second disc 126 with respect to the slide assembly 112 and allow for rotation of the second disc 126 to be transmitted to the slide assembly 112, the slip clutch assembly further includes a driveshaft 130 fixed to the threaded shaft 114 of the slide assembly 112 and extending towards the knob assembly 106. The driveshaft 130 includes a plurality of splines 132. Referring also to FIG. 7, the second disc 126 is annular and includes an inner surface that has a plurality of longitudinal grooves 134 (only two of which are labelled). The second disc 126 is received on the driveshaft 130 with the splines 132 engaged with the grooves 134. In order to move between the first position and the second position, the second disc 126 slides along the driveshaft 132. Furthermore, due to the engagement of the splines 132 and the grooves 134, rotation of the second disc 126 causes rotation of the threaded shaft 114.

Referring still to FIG. 6, in the example shown, the slip clutch assembly 120 further includes a biasing member in the form of a spring 136, more specifically a compression spring. The spring 136 biases the second disc 126 towards the first position, while allowing movement of the second disc 126 to the second position. In the example shown, the slip clutch assembly 120 includes a flange 138 from which the driveshaft 130 extends, and the spring 136 is received on the driveshaft 130 between the flange 138 and the second disc 126, so that it is compressed between the flange 138 and the second disc 126.

Referring back to FIG. 3 in use, the spring 136 forces the second disc 126 to the first position, so that the second engagement face 128 (shown in FIG. 6) is maintained in engagement with the first engagement face 124 (shown in FIG. 6). This engagement couples the knob assembly 106 to the slide assembly 112. If torque is applied to the knob assembly 106 to rotate the knob assembly 106, the rotation of the knob assembly 106 will be transmitted to the slide assembly 112 via the slip clutch assembly 120, to cause rotation of the threaded shaft 114. That is, rotation of the knob assembly 106 will be transmitted to the first disc 122, rotation of the first disc 112 will be transmitted to the second disc 126, rotation of the second disc 126 will be transmitted to the driveshaft 130, and rotation of the driveshaft 130 will be transmitted to the threaded shaft 114 of the slide assembly 112. This in turn will cause translation of the slider 116, tensioning of the control wire 118, and deflection of the tool 104 (not shown in FIG. 3).

Referring next to FIG. 4, if tension on the control wire 118 (not shown in FIG. 4) increases (as might occur if, for example, a stiff secondary tool is within the tool 104 or if the tool 104 is in contact with patient anatomy) and the torque applied to the knob assembly 106 is increased to above a threshold value (as might occur if, for example, a user attempts to force the rotation of the knob assembly 106 despite the tension on the control wire 118), the knob assembly 106 will decouple from the slide assembly 112, via the slip clutch assembly 120. More specifically, torque applied to the knob assembly 106 will cause rotation of the first disc 122; however, due to the tension on the control wire 118, the threaded shaft 114 will resist rotation. Due to the inclined side surfaces of the first and second sets of teeth, rotation of the first disc 122 will cause movement the second disc 126 away from the first disc 122—i.e. the second disc 126 will compress the spring 136 and translate along the driveshaft 130, to disengage the first engagement face 124 (shown in FIG. 6) and the second engagement face 128 (shown in FIG. 6).

Referring next to FIG. 5, if tension on the control wire 118 (not shown in FIG. 5) decreases (as might occur if, for example, the stiff secondary tool is removed from within the tool 104) and the torque applied to the knob assembly 106 is decreased to below the threshold value, the knob assembly 106 will recouple to the slide assembly 112 via the slip clutch assembly 120. More specifically, the spring 136 will bias the second disc 126 towards the first disc 122 and back to the first position, to re-engage the first engagement face 124 (shown in FIG. 6) and the second engagement face 128 (shown in FIG. 6). When the second disc 126 is back in the first position, rotation of the knob assembly 106 will be transmitted to the first disc 122, rotation of the first disc 122 will be transmitted to the second disc 126, rotation of the second disc 126 will be transmitted to the driveshaft 130, and rotation of the driveshaft 130 will be transmitted to the threaded shaft 114 of the slide assembly 112. This in turn will cause translation of the slider 116, tensioning of the control wire 118, and deflection of the tool 104 (not shown in FIG. 5).

In the above examples, the slip clutch assembly 120 may optionally provide tactile and/or auditory feedback to a user, to indicate to the user that the control wire 118 is under tension. For example, as the second disc 126 moves to the second position, the user may feel a change in the resistance to rotation of the knob assembly 106. Alternatively or in addition, an audible or tactile click may be felt when the second disc 126 moves back to the first position. Alternatively or in addition, the handle 102 may be provided with a visual indicator (e.g. a window that allows the user to see disengagement of the first disc 122 and the second disc 126) to indicate to the user that the control wire 118 is under tension.

In alternative examples, rather than providing only binary coupling and decoupling of the knob assembly 106 and the slide assembly 112, the slip clutch assembly 120 can be configured to provide intermediate levels of coupling. For example, the teeth geometry and spring can be selected so that for a range of torques applied to the knob assembly 106, the slip clutch assembly 120 transmits some rotation before decoupling the knob assembly 106 and the slide assembly 112. This can serve as an alerting system, to alert the user that the torque is approaching the threshold value.

In alternative examples, rather than or in addition to being used to control tension on the control wire 118, the slip clutch assembly 120 can be used to limit the amount of deflection that the user can impart to the tool 104.

In alternative examples, the slip clutch assembly 120 may be configured so that the first disc 122 is movable towards and away from the knob assembly 106, and the second disc 126 is translationally fixed. That is, either the first disc 122 or the second disc 126 may be translatable between a first position and a second position.

In alternative examples, rather than relying on mechanical engagement of the first engagement face and the second engagement face (e.g. engagement using teeth), the first engagement face and second engagement face can be magnetically attracted to each other in order to transmit rotation from the first disc to the second disc. The magnets can be selected such that when the torque is below the threshold value, the second disc rotates with the first disc due to magnetic attraction; however, when the torque is above the threshold value, the first disc rotates without transmitting rotation to the second disc. Alternatively, the first engagement face and second engagement face can rely on frictional forces between the two faces in order to transmit rotation from the first disc to the second disc. In such examples, a biasing member can be omitted, and the second disc can be longitudinally fixed with respect to the driveshaft and with respect to the first disc.

In any of the above examples, a lubricant may be provided to facilitate movement of the various parts.

The term ‘disc’ is used herein for simplicity, and is not limited to any particular shape. That is, a disc may be circular or another shape.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A steerable medical device comprising:
a handle having a rotatable knob assembly and housing a slide assembly, wherein the knob assembly is couplable to the slide assembly to drive movement of the slide assembly by rotation of the knob assembly;
an elongate tool extending from the handle;
at least one control wire coupled between the slide assembly and the tool, whereby movement of the slide assembly causes tensioning of the control wire, and tensioning of the control wire causes deflection of the tool; and
a slip clutch assembly between the knob assembly and the slide assembly, whereby when torque applied to the knob assembly is below a threshold value, the slip clutch assembly couples the knob assembly to the slide assembly to drive movement of the slide assembly by rotation of the knob assembly, and whereby when torque applied to the knob assembly is above the threshold value, the slip clutch assembly decouples the knob assembly from the slide assembly to prevent movement of the slide assembly by rotation of the knob assembly.

2. The steerable medical device of claim 1, wherein the slip clutch assembly comprises:
a first disc rotatable with the knob assembly and having a first engagement face; and
a second disc configured to transmit rotation to the slide assembly and having a second engagement face.

3. The steerable medical device of claim 2, wherein at least one of the first disc and the second disc is translatable between a first position and a second position, wherein in the first position the first engagement face and the second engagement face are engaged to couple the rotatable knob to the slide assembly, and in the second position the first engagement face and the second engagement face are disengaged to decouple the rotatable knob from the slide assembly.

4. The steerable medical device of claim 3, wherein the first disc is fixed with respect to the knob assembly, and the second disc is coupled to the slide assembly and is translatable between the first position and the second position.

5. The steerable medical device of claim 4, wherein:
the slip clutch assembly further comprises a driveshaft fixed to the slide assembly and extending towards the rotatable knob. and a spring biasing the second disc towards the first position;
the driveshaft comprises a plurality of splines;
the second disc is annular and comprises an inner surface having a plurality of longitudinal grooves; and
the second disc is received on the drive shaft with the splines engaged with the grooves.

6. The steerable medical device of claim 5, wherein the slip clutch assembly further comprises a flange from which the driveshaft extends, wherein the spring is received on the driveshaft and is compressed between the flange and the second disc.

7. The steerable medical device of claim 3, wherein the first engagement face has a first set of teeth, and the second engagement face has a second set of teeth, and wherein the first set of teeth and the second set of teeth each have inclined side surfaces to facilitate movement from the first position to the second position by rotation of the first disc.

8. The steerable medical device of claim 2, wherein the second disc is longitudinally fixed with respect to the first disc.

9. The steerable medical device of claim 2, wherein when torque applied to the knob assembly is below a threshold value, the first disc transmits rotation to the second disc by magnetic forces or frictional forces between the first engagement face and the second engagement face.

10. A handle for a medical device, the handle comprising:
a rotatable knob assembly and a slide assembly, wherein the knob assembly is couplable to the slide assembly to drive movement of the slide assembly by rotation of the knob assembly;
a slip clutch assembly between the knob assembly and the slide assembly, the slip clutch assembly comprising a first disc and a second disc, wherein the first disc has a first engagement face and is rotatable with the knob assembly, wherein the second disc has a second engagement face and is configured to transmit rotation to the slide assembly, wherein the first engagement face and the second engagement face are engageable to couple the rotatable knob to the slide assembly to drive movement of the slide assembly by rotation of the knob assembly, and are disengageable to decouple the rotatable knob and the slide assembly to prevent movement of the slide assembly by rotation of the knob assembly.

11. The handle of claim 10, wherein at least one of the first disc and the second disc is translatable with respect to the slide assembly between a first position and a second position, wherein in the first position the first engagement face and the second engagement face are engaged to couple the rotatable knob to the slide assembly, and in the second position the first engagement face and the second engagement face are disengaged to decouple the rotatable knob from the slide assembly.

12. The handle of claim 11, wherein the first disc is fixed to the knob assembly, and the second disc is coupled to the slide assembly and is translatable between the first position and the second position.

13. The handle of claim 12, wherein:
the slip clutch assembly further comprises a driveshaft fixed to the slide assembly and extending towards the knob assembly, and a spring biasing the second disc toward the first position;
the driveshaft comprises a plurality of splines;
the second disc is annular and comprises an inner surface having a plurality of longitudinal grooves; and
the second disc is received on the drive shaft with the splines engaged with the grooves.

14. The handle of claim 13, wherein the slip clutch assembly further comprises a flange from which the driveshaft extends, wherein the spring is received on the driveshaft and is compressed between the flange and the second disc.

15. The handle of claim 11, wherein the first engagement face has a first set of teeth and the second engagement face has a second set of teeth, and wherein the first set of teeth and the second set of teeth each have inclined side surfaces to facilitate translation from the first position to the second position by rotation of the first disc.

16. The handle of claim 10, wherein the second disc is longitudinally fixed with respect to the first disc.

17. The handle of claim 10, wherein the first engagement face and second engagement face are engageable by magnetic forces or frictional forces.

18. A method for operating a medical device, comprising:
a. with a knob assembly of the medical device coupled to a slide assembly of the medical device via a slip clutch assembly, applying torque to the knob assembly to rotate the knob assembly, wherein the slip clutch assembly comprises a first disc having a first engagement face and a second disc having a second engagement face engageable with the first engagement face, wherein the first disc is rotatable with the knob assembly, and the second disc is configured to transmit rotation to the slide assembly; and
b. transmitting rotation of the knob assembly to the slide assembly via the slip clutch assembly, including transmitting rotation of the knob assembly to the first disc, transmitting rotation of the first disc to the second disc, and transmitting rotation of the second disc to the slide assembly.

19. The method of claim 18, wherein in step b., the second engagement face is maintained in engagement with the first engagement face by a spring.

20. The method of claim 18, further comprising:
c. increasing the torque applied to the knob assembly to exceed a threshold value; and
d. as a result of step c., decoupling the knob assembly from the slide assembly via the slip clutch assembly.
